(19) United States
(12) Patent Application Publication
(10) Pub. No.: US 2019/0330640 A1
(43) Pub. Date: Oct. 31, 2019

(54) OLIGONUCLEOTIDES INHIBITING THE EXPRESSION OF NRP1

(71) Applicant: SECARNA PHARMACEUTICALS GMBH & CO. KG, Marburg (DE)

(72) Inventors: Frank JASCHINSKI, Puchheim (DE); Tamara HILMENYUK, Munich (DE); Sven MICHEL, Bernried (DE)

(21) Appl. No.: 16/476,741

(22) PCT Filed: Jan. 9, 2018
(86) PCT No.: PCT/EP2018/050440

§ 371 Date: Jul. 9, 2019

(30) Foreign Application Priority Data
Jan. 9, 2017 (EP) 17150687.6

(51) Int. Cl. C12N 15/113; A61K 31/7125; A61P 27/02; A61P 35/00

(52) U.S. Cl. CPC C12N 15/1138; A61K 31/7125; A61K 45/06; A61P 35/00; A61P 27/02

(57) ABSTRACT

The present invention refers to an oligonucleotides comprising 12 to 18 nucleotides, wherein at least one of the nucleotides is modified, and the oligonucleotide hybridizes with a nucleic acid sequence of neuropilin 1 (NRP1, CD304) of SEQ ID NO.1 (NM_003873.5), wherein the oligonucleotide inhibits at least 50% of the NRP1 expression. The invention is further directed to a pharmaceutical composition comprising such oligonucleotide.

The present invention refers to an oligonucleotide hybridizing with a nucleic acid sequence of neuropilin (NRP) such as NRP1 (CD304) and a pharmaceutical composition comprising such oligonucleotide and a pharmaceutically acceptable carrier, excipient and/or diluent.

### TECHNICAL BACKGROUND

Neuropilin 1 (NRP1) is a multi-domain receptor involved in versatile signal transduction pathways that control cell migration, angiogenesis, cell survival, metastasis, and cell proliferation. Accordingly, NRP1 has been shown to serve as a co-receptor for a broad spectrum of growth factor receptors (Prud'homme et al., Oncotarget, 2012, 3(9): 921-939). Common binding partners for NRP1 are for example TGF-beta receptor I and II; vascular endothelial growth factor receptor (VEGFR), platelet derived growth factor receptor (PDGFR) and plexin (semaphorin receptor) (Prud'homme et al., Oncotarget, 2012, 3(9): 921-939). NRP1 expression has been reported in a wide variety of cells including cells of the immune system such as regulatory T cells (T_{regs}) (Prud'homme et al., Oncotarget, 2012, 3(9): 921-939). In general, neuropilins are overexpressed in several human tumor types, including carcinomas, melanoma, glioblastoma, leukemias and lymphomas. Overexpression of NRP1 correlates with more aggressive clinical tumor behavior (Prud'homme et al., Oncotarget, 2012, 3(9): 921-939).

NRP1 has also been shown to be involved in the maturation of blood vessels. Former studies indicate that immature blood vessels depend on and respond to vascular endothelial growth factor (VEGF), which is a common ligand of NRP1 (Pan et al., Cancer Cell, 2007, 11(1), 53-67). Accordingly, inhibiting NRP1 expression would prevent abnormal blood vessel maturation and force them into an immature, VEGF dependent state. Therefore, a combined anti-NRP1 and anti-VEGF therapy is beneficial for the treatment of angiogenesis related ophthalmologic diseases and tumors.

Functionally, NRP1 has been linked to immune inhibition. Several studies indicate that regulatory T cells (T_{regs}) express NRP1 on their surface (Hansen, W., Oncoimmunology, 2013, 2(2), e230399). VEGF producing tumor cells attract the NRP1-expressing T_{regs} due to the interaction of NRP1 and VEGF. While VEGF promotes tumor angiogenesis, the T_{regs} interfere with anti-tumor immune responses, e.g. by secreting immunosuppressive cytokines (Hansen, et al., Oncoimmunology, 2013, 2(2), e230399). Inhibition of NRP1 would prevent the infiltration of T_{regs} in the tumor microenvironment and therefore improve anti-tumor immune responses.

Few antibodies have been developed to inhibit activity of NRP1: The anti-human NRP1 monoclonal antibody MNRP1685A (Genentech) inhibits specifically the VEGF binding domain of NRP1. This antibody was used in phase I clinical studies to treat patients suffering from advanced solid tumors (Weekes, et al., Investigational New Drug, 2014, 32(4), 653-660). However, relatively high concentrations and repetitive dosing of the antibody is needed to successfully block NRP1.

Furthermore, the anti-VEGF antibody Aflibercept (Sanofi) is used as common therapy to treat diseases with pathological retinal angiogenesis, e.g. neovascular (wet) age-related macular degeneration (AMD), diabetic retinopathy and retinopathy of prematurity. However, application of Aflibercept is limited as it cannot inhibit binding of non-classical ligands to NRP1 which act as pro-angiogenic growth factors (e.g., TGF-beta, PDGF, semaphorines, HGF) and it shows only low activity against matured blood vessels. Relatively high concentrations and repetitive dosing via monthly intravitreal injections are required to successfully block NRP1 activity. As these therapies are very inconvenient for the patient, there is a need to develop improved therapies that enable less frequent applications.

Furthermore, one small molecule EG00229 (Tocris) acts as a receptor antagonist of NRP1. EG00229 has been shown to inhibit binding of VEGF-A to the b1 domain of NRP1 at least in vitro. EG00229 enhances the chemo-sensitivity of A549 cells (Jarvis et al., Journal of Medicinal Chemistry, 2010, 53(5), 2215-2226), however, its clinical efficacy has not been determined in vivo so far.

U.S. Pat. No. 7,087,580 refers to oligonucleotides hybridizing with human neuropilin 1 comprising first generation modifications and mutations such as substitutions, insertions and deletions.

NRP1 comprises several, partially overlapping binding sites for different ligands and co-receptors. Common approaches using a single antibody, first generation oligonucleotide and/or a small molecule cannot or hardly Nock all interactions sites of such a multi-domain receptor. Antibody based therapies would require administering more than one antibody. Accordingly, an agent which is safe and effective in inhibiting simultaneously the complete functions mediated by a receptor such as NRP1 would be an important addition for the treatment of patients suffering from diseases or conditions affected for example by the activity of NRP1 and its pro-angiogenic ligands.

Oligonucleotides of the present invention are very successful in the inhibition of the expression and activity of NRP1, respectively. The mode of action of an oligonucleotide differs from the mode of action of an antibody or small molecule, and oligonucleotides are highly advantageous regarding for example

(i) the Mocking of multiple functions and activities, respectively, of a target,
(ii) the penetration of tumor tissue in solid tumors due to their small molecular size,
(iii) the combination of oligonucleotides with each other or an antibody or a small molecule, and
(iv) the inhibition of intracellular effects which are not accessible for an antibody or inhibitable via a small molecule.

Oligonucleotides of the present invention are advantageous in comparison to first generation oligonucleotides due to their higher stability, stronger target affinity and potency and due to their independence from delivery reagents to achieve target suppression in cells.

### SUMMARY

The present invention refers to an oligonucleotide comprising about 12 to 18 nucleotides, wherein at least one of the nucleotides is modified. The oligonucleotide hybridizes for example with a nucleic acid sequence of human neuropilin 1 (NRP1, CD304) of SEQ ID NO.1 (NM_003873.5). Furthermore, the oligonucleotide is cross-reactive with the corresponding mouse and rat sequences. The modified nucleotide is for example selected from the group consisting of a bridged nucleic acid (e.g., LNA, cET, ENA, 2′Fluoro modified nucleotide, 2′O-Methyl modified nucleotide or a combination thereof). The oligonucleotide of the present invention inhibits for example at least 50%, at least 60%, at least 70%, at least 80%, at least 90% or at least 99% of the NRP1 expression for example 50 to 99%, 55 to 95%, 60% to 90%, or 65 to 85%. The oligonucleotide of the present invention inhibits for example the expression of NRP1 at a nanomolar concentration.

The present invention is further directed to a pharmaceutical composition comprising an oligonucleotide of the present invention and optionally a pharmaceutically acceptable carrier, excipient, diluent or a combination thereof. In some embodiments, this pharmaceutical composition additionally comprises a chemotherapeutic, another oligonucleotide, an antagonistic protein such as a fusion protein, an antibody and/or a small molecule which is for example effective in tumor treatment or in treatment of an ophthalmic disease.

In some embodiments, the oligonucleotide of the present invention is in combination with another oligonucleotide, an antagonistic protein such as a fusion protein, an antibody and/or a small molecule, either each of these compounds separate or combined in a pharmaceutical composition, wherein the oligonucleotide of the present invention inhibits the activity of a receptor such as a growth receptor selected from the group consisting of TGF-beta receptor I (TβRI), TGF-beta receptor II (TβRII), receptors for VEGF, HGF, PDGF and SEMA3 (Plexin), or a combination thereof. In some embodiments, the oligonucleotide of the present invention is in combination with another oligonucleotide, an antagonistic protein such as a fusion protein, an antibody and/or a small molecule, either each of these compounds separate or combined in a pharmaceutical composition, wherein the oligonucleotide of the present invention inhibits the activity of a signal transduction factor such as p38MAPK, ERK1, ERK2, PI3K, Akt, NF-κB, pSMAD2, pSMAD3, Src, Pyk2, FAK, p-p130Cas, or a combination thereof.

In some embodiments, the present invention relates to the pharmaceutical composition of the present invention, wherein another oligonucleotide, an antagonistic protein such as a fusion protein, the antibody and/or the small molecule inhibits the identical or a different growth receptor or signal transduction factor than the antisense oligonucleotide according to the present invention.

Furthermore, the present invention relates to an oligonucleotide or a pharmaceutical composition of the present invention for inhibiting the immigration of a Tre_{g} cell into a tumor.

Furthermore, the present invention relates to the use of the oligonucleotide or the pharmaceutical composition of the present invention in a method of preventing and/or treating a cancer, an ophthalmic disease, an autoimmune disorder and/or an immune disorder. In some embodiments, the disorder is for example an angiogenic eye disease such as age related macular disease (AMD), diabetic retinopathy (DME), retinopathy of prematurity (Retinopathia praematurorum) or corneal neovascularization (nv), e.g., deep nv overlying Descemet's membrane seen for example in herpetic and syphilitic stromal keratitis; stromal nv for example associated with (most) forms of stromal keratitis; and vascular pannus which is for example composed of connective tissue proliferating in the superficial corneal periphery and for example associated with ocular surface disorders. In some embodiments, the oligonucleotide or the pharmaceutical composition of the present invention is for example administered locally or systemically.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIG. 1 shows the mRNA sequence of human (h) NRP1 (SEQ ID No. 1; reference NM_003873.5).

FIG. 2 depicts the distribution of hmr (human-, mouse-, and rat-cross-reactive) NRP1 antisense oligonucleotide binding sites on the hNRP1 mRNA of SEQ ID No. 1 (NM_003873.5) as well as their modification(s) and length. hmrNRP1 antisense oligonucleotides were aligned to the hNRP1 mRNA sequence of SEQ ID No. 1. The different grayscales indicate the different LNA modifications and symbols indicate the different length of the antisense oligonucleotides.

FIG. 3A and 3B depict hmrNRP1 mRNA knockdown efficacy of hmrNRP1 antisense oligonucleotides in a human cancer cell line SKOV-3 (human ovary adenocarcinoma; FIG. 3A) and a mouse cancer cell line Renca (Renal cell carcinoma; FIG. 3B). SKOV-3 and Renca cells were treated for 3 days with 10 μM of the respective antisense oligonucleotide. As negative control, cells were either treated with negl, an antisense oligonucleotide having the sequence CGTTTAGGCTATGTACTT (described in WO2014154843 A1). Residual human and mouse NRP1 mRNA expression, respectively, relative to untreated cells is depicted. Expression values were normalized to expression of the housekeeping gene HPRT1. FIG. 3C depict the viability of mouse Renca cells after treatment with hmrNRP1 antisense oligonucleotides as determined by a cell titer blue assay.

FIG. 4 shows a correlation analysis of the efficacy of NRP1 antisense oligonucleotides in SKOV-3 and Renca cells.

FIG. 5 shows concentration-dependent mNRP1 mRNA knockdown by selected hmrNRP1 antisense oligonucleotides in Renca cells which were A15001HMR (SEQ ID No. 3) and A15005HMR (SEQ ID No. 2). Renca cells were treated for 3 days with the indicated concentration of the respective antisense oligonucleotide. Residual mNRP1 expression is depicted compared to untreated control cells. mNRP1 mRNA expression values were normalized to expression of the housekeeping gene HPRT1. Concentration-dependent target knockdown was used for calculation of IC50 values shown in Table 4.

FIG. 6A depicts concentration dependent hNRP1 protein knockdown by A15001HMR, (SEQ ID No. 3), A15005HMR (SEQ ID No. 2), A15006HMR (SEQ ID No. 4), A15008HMR (SEQ ID No. 5), A15011HMR (SEQ ID No. 6). Analysis of NRP1 protein expression by flow cytometry in SKOV-3 cells was performed after treatment with the indicated antisense oligonucleotides for 3+3 days. As a control, cells were treated with Scrambled 6 (S6; SEQ ID No. 28) for 3+3 days at the indicated concentrations. Relative expression compared to untreated cells (set as 1) is shown. FIG. 6B depicts the effect on viability of the oligonucleotide treatment compared to untreated cells as determined by 7-AAD staining using flow cytometry.

FIG. 7A depicts concentration-dependent mNRP1 protein knockdown by A15005HMR (SEQ ID No. 2). Analysis of mNRP1 protein expression by flow cytometry in Renca cells was performed after treatment with the indicated antisense oligonucleotides for 3 days. As treatment control, cells were treated with Scrambled 6 (S6; SEQ ID No. 28) for 3 days at the indicated concentrations. Relative expression compared to untreated control cells (set as 1) is depicted. FIG. 7B depicts the effect on viability of oligonucleotide treatment compared to untreated cells as determined by 7-AAD staining using flow cytometry.

FIGS. 8A and 8B show the general domain structure of neuropilin and hypothetical model of interaction with multiple growth factors (see Prud'homme G and Glinka Y, Oncotarget 2012, 3: 921-939).

FIG. 9 depicts NRP1 mRNA expression levels in retinae from C57BL/6 mice 3 or 10 days after single intravitreal injections of either A15005HMR (SEQ ID No. 2) or negative control oligonucleotide S5 (SEQ ID No. 29). Expression values were normalized to expression values of the housekeeping gene HPRT1.

FIG. 10 depicts NRP1 mRNA expression levels in retinae from C57BL/6 mice 24 days after treatment with single intravitreal injection of either A15005HMR (SEQ ID No. 2) or the negative control oligonucleotide S5 (SEQ ID No. 29; 16-18 eyes/group). Expression values were normalized to expression values of the housekeeping gene HPRT1.

### DETAILED DESCRIPTION

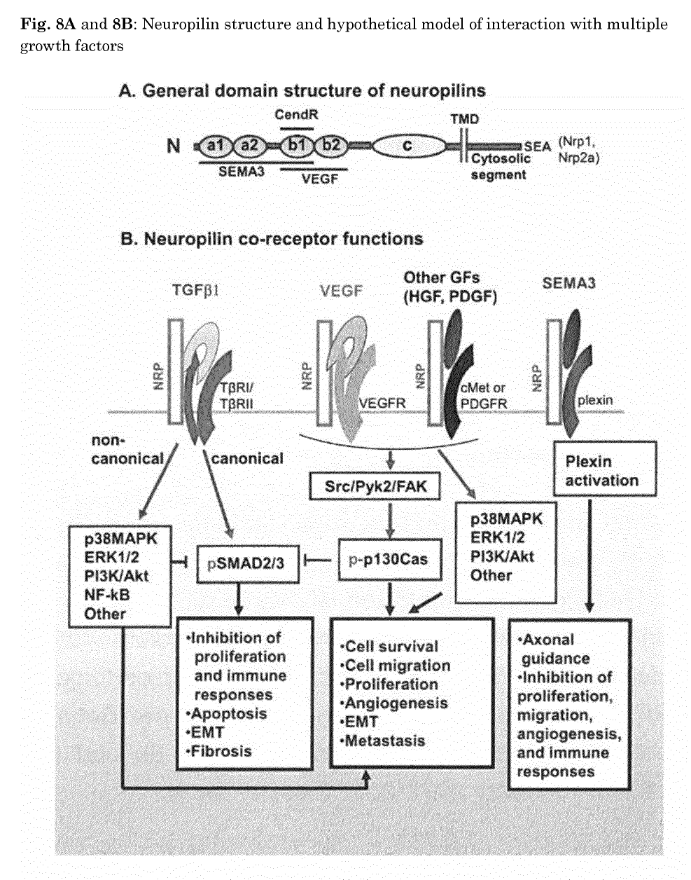

The present invention provides human-, murine- and rat-specific oligonucleotides which hybridize with mRNA sequences of neuropilin such as NRP1 of human mouse and/or rat and inhibit the expression and activity, respectively, of NRP1. NRP1 as a multi-domain receptor binds to several different types of ligands and receptors relevant for cell migration, angiogenesis, cell survival, metastasis and cell proliferation (see FIG. 8). Many ligands of NRP1 act as pro-angiogenic growth factors. Therefore, inhibition of expression of NRP1 allows to target a broad spectrum of different activities of NRP1 simultaneously, thereby significantly increasing the feasibility of a successful therapy. Thus, the present invention refers to oligonucleotides inhibiting at least 50% of the NRP1 expression. They represent an interesting and highly efficient tool for use in a method of preventing and/or treating cancer, an ophthalmic disease, an autoimmune disorder and/or an immune disorder.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides consisting of or comprising 10 to 25 nucleotides, 10 to 15 nucleotides, 15 to 20 nucleotides, 12 to 18 nucleotides, or 14 to 17 nucleotides. The oligonucleotides for example consist of or comprise 10, 11, 12, 13, 14, 15, 16, 17, 18, 19, 20 or 25 nucleotides. The oligonucleotides of the present invention comprise for example at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide or a combination thereof. In some embodiments, the oligonucleotide of the present invention comprises nucleotides having the same or different modifications. The oligonucleotide of the present invention in addition comprises for example a modified phosphate backbone, wherein the phosphate is for example a phosphorothioate.

The oligonucleotide of the present invention comprises the one or more modified nucleotide at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more unmodified nucleotides. The following Table 1 presents embodiments of oligonucleotides comprising modified nucleotides for example LNA which are indicated by (+) and phosphorothioate (PTO) indicated by (*). The oligonucleotides consisting of or comprising the sequences of Table 1 may comprise any other modified nucleotide and any other combination of modified and unmodified nucleotides. Oligonucleotides of Table 1 hybridize with mRNA of human, mouse and rat NRP1:

TABLE 1
List of antisense oligonucleotides hybridizing with human, mouse and
rat NRP1 for example of SEQ ID No. 1; Neg1 is an antisense
oligonucleotide representing a negative control which is
not hybridizing with NRP1 of SEQ ID No. 1. S5 and S6 are
control antisense oligonucleotides having no sequence
complementarity to any human or mouse mRNA.

| SEQ | Name | | |
|---|---|---|---|
| ID | (Compound | mRNA (Antisense) Sequence | Antisense Sequence 5′-3′ with |
| No. | ID) | 5′-3′ | PTO (*) and LNA (+) |
| 2 | A15005HMR | GTCTCAAGTCGCCTG | +G*+T*+C*T*C*A*A*G*T*C*G*C*+C*+T*+G |
| 3 | A15001HMR | AAGTCGCCTGCATC | +A*+A*G*T*C*G*C*C*T*G*C*+A*+T*+C |
| 4 | A15006HMR | GGACTTTATCACTCC | +G*G*A*C*T*T*T*A*T*C*A*C*+T*C*+C |
| 5 | A15008HMR | ATCCTGTCATTTAGCT | +A*+T*+C*C*T*G*T*C*A*T*T*T*A*+G*+C*+T |
| 6 | A15011HMR | TCGGACAAATCGAGTT | +T*+C*+G*G*A*C*A*A*A*T*C*G*A*+G*+T*+T |
| 7 | A15002HMR | ATCGAGTTATCAGT | +A*+T*+C*G*A*G*T*T*A*T*C*A*+G*+T |
| 8 | A15003HMR | GTTGGCCTGGTCGT | +G*T*T*G*G*C*C*T*G*G*T*+C*G*+T |
| 9 | A15004HMR | GTTAAGGCTTCGCT | +G*+T*T*A*A*G*G*C*T*T*C*+G*C*+T |
| 10 | A15007HMR | TCGGACAAATCGAGT | +T*C*+G*G*A*C*A*A*A*T*C*G*+A*+G*+T |
| 11 | A15009HMR | CGCCTGCATCCTGTCA | +C*+G*+C*C*T*G*C*A*T*C*C*T*G*+T*+C*+A |
| 12 | A15010HMR | AGTCGCCTGCATCCTG | +A*+G*+T*C*G*C*C*T*G*C*A*T*C*+C*+T*+G |
| 13 | A15012HMR | TTCGGACAAATCGAGT | +T*+T*+C*G*G*A*C*A*A*A*T*C*G*+A*G*+T |
| 14 | A15013HMR | AATAAGTCCAGACACC | +A*+A*+T*A*A*G*T*C*C*A*G*A*C*+A*+C*+C |
| 15 | A15014HMR | TACTGTCACTTTAACC | +T*+A*+C*T*G*T*C*A*C*T*T*T*A*+A*+C*+C |
| 16 | A15015HMR | GAAGCAATAATATACC | +G*+A*+A*G*C*A*A*T*A*A*T*A*T*+A*+C*+C |
| 17 | A15016HMR | GGCTTCGCTGTTCATT | +G*+G*+C*T*T*C*G*C*T*G*T*T*C*+A*+T*+T |
| 18 | A15017HMR | TAAGGCTTCGCTGTTC | +T*+A*+A*G*G*C*T*T*C*G*C*T*G*+T*+T*+C |
| 19 | A15018HMR | TTAAGGCTTCGCTGTT | +T*+T*+A*A*G*G*C*T*T*C*G*C*T*+G*+T*+T |
| 20 | A15019HMR | CGCCTGCATCCTGTCAT | +C*+G*+C*C*T*G*C*A*T*C*C*T*G*T*+C*+A*+T |
| 21 | A15020HMR | TCGCCTGCATCCTGTCA | +T*+C*+G*C*C*T*G*C*A*T*C*C*T*G*T*+C*+A |
| 22 | A15021HMR | TCGCCTGCATCCTGTCA | +T*+C*G*C*C*T*G*C*A*T*C*C*T*G*T*C*+A |
| 23 | A15022HMR | GTCTCAAGTCGCCTGCA | +G*+T*+C*T*C*A*A*G*T*C*G*C*C*T*+G*+C*+A |
| 24 | A15023HMR | TTCGGACAAATCGAGTT | +T*+T*+C*G*G*A*C*A*A*A*T*C*G*A*+G*+T*+T |
| 25 | A15024HMR | CGATCTTGAACTTCCTC | +C*+G*+A*T*C*T*T*G*A*A*C*T*T*C*+C*+T*+C |
| 26 | A15025HMR | TGGCAGTTGGCCTGGTC | +T*+G*+G*C*A*G*T*T*G*G*C*C*T*G*G*+T*+C |
| 27 | neg1 | CGTTTAGGCTATGTACTT | +C*+G*+T*T*T*A*G*G*C*T*A*T*G*T*A*+C*+T*+T |
| 28 | S6 | TCTATCGTGATGTTTCT | +T*+C*+T*A*T*C*G*T*G*A*T*G*T*T*+T*+C*+T |
| 29 | S5 | TTATGTCCGGTTATTTC | +T*+T*+A*T*G*T*C*C*G*G*T*T*A*T*+T*+T*C |

The oligonucleotides of the present invention hybridize for example with mRNA of human, murine or rat NRP of SEQ ID No. 1. Such oligonucleotides are called NRP antisense oligonucleotides. The oligonucleotides hybridize for example within position 303 and 5730 of NRP1 mRNA of SEQ ID No. 1.

In some embodiments, the oligonucleotide of the present invention inhibits at least about 50%, 55%, 60%, 65%, 70%, 75%, 80%, 85%, 90%, 92%, 94%, 95%, 96%, 97%, 98%, 99% or 100% of NRP such as the, e.g., human, rat or murine, NRP1 expression. Thus, the oligonucleotides of the present invention are oligonucleotides which inhibit expression and activity of NRP1 for example in a cell, tissue, organ, or a subject. The oligonucleotide of the present invention inhibits the expression of NRP such as NRP1 at a nanomolar or micromolar concentration for example in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is for example used in a concentration of 1, 3, 5, 9, 10, 15, 27, 30, 40, 50, 75, 82, 100, 250, 300, 500, or 740 nM, or 1, 2.2, 3, 5, 6.6 or 10 μM.

In some embodiments the present invention refers to a pharmaceutical composition comprising an oligonucleotide of the present invention and a pharmaceutically acceptable carrier, excipient and/or diluent. The pharmaceutical composition further comprises for example a chemotherapeutic, another oligonucleotide either from the present invention or different from the present invention, an antagonistic protein such as a fusion protein, an antibody and/or a small molecule.

In some embodiments, the oligonucleotide or the pharmaceutical composition of the present invention is for use in a method of preventing and/or treating a disorder. In some embodiments, the use of the oligonucleotide or the pharmaceutical composition of the present invention in a method of preventing and/or treating a disorder is combined with radiotherapy and/or laser treatment. The radiotherapy may be further combined with a chemotherapy (e.g., platinum, gemcitabine). The disorder is for example characterized by an NRP imbalance, i.e., the NRP level is increased in comparison to the level in a normal, healthy cell, tissue, organ or subject. The NRP level is for example increased by an increased NRP such as NRP1 expression and activity, respectively. The NRP level can be measured by any standard method such as immunohistochemistry, western blot, flow cytometry, quantitative real time PCR or QuantiGene assay known to a person skilled in the art.

The oligonucleotide and the pharmaceutical composition comprising the oligonucleotide, respectively, of the present invention has an inhibitory effect on the NRP1 expression for example for 1, 2, 3, 4, 5 or 6 days, 1, 2 or 3 weeks, 1, 2, 3, 4, 5, 6, 7, 8, 9, 10, 11 months or 1 or 2 years. The treatment effect of the oligonucleotides of the present invention for example corresponds to the duration of the inhibitory effect.

An oligonucleotide or a pharmaceutical composition of the present invention is administered locally or systemically for example intravitreal, intracameral or subconjunctival, e.g., injection, topically via eye drops, orally, sublingually, nasally, subcutaneously, intravenously, intraperitoneally, intramuscularly, intratumorally, intrathecal, transdermally, and/or rectally. Alternatively or in combination ex vivo treated immune cells are administered. The oligonucleotide is administered alone or in combination with another oligonucleotide of the present invention and optionally in combination with another compound such as another oligonucleotide, an antagonistic protein such as a fusion protein, an antibody, a small molecule and/or a chemotherapeutic (e.g., platinum, gemcitabine). In some embodiments, the other oligonucleotide (i.e., not being part of the present invention), the antagonistic protein such as a fusion protein, the antibody, and/or the small molecule are effective in preventing and/or treating cancer, an ophthalmic disease, an autoimmune disorder and/or an immune disorder. An oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are bladder carcinoma, breast cancer, colorectal carcinoma, lung cancer, malignant melanoma, mesothelioma, lymphoma, skin cancer, bone cancer, prostate cancer, hepatocarcinoma, brain cancer, cancer of the larynx, liver, gall bladder, pancreas, testicular, rectum, parathyroid, thyroid, adrenal, neural tissue, head and neck, colon, stomach, bronchi, kidneys, basal cell carcinoma, neuroblastoma, squamous cell carcinoma, metastatic skin carcinoma, osteo sarcoma, Ewing's sarcoma, reticulum cell sarcoma, liposarcoma, leukemia, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythaemia vera, adenocarcinoma, anaplastic astrocytoma, glioblastoma multiforma, leukemia, or epidermoid carcinoma.

In some embodiments two or more oligonucleotides of the present invention are administered together, at the same time point for example in a pharmaceutical composition or separately, or on staggered intervals. In other embodiments, one or more oligonucleotides of the present invention are administered together with another compound such as another oligonucleotide (i.e., not being part of the present invention), an antagonistic protein such as a fusion protein, an antibody, a small molecule and/or a chemotherapeutic, at the same time point for example in a pharmaceutical composition or separately, or on staggered intervals. In some embodiments of these combinations, the oligonucleotide inhibits the expression and activity, respectively, of an receptor such as an growth receptor and the other oligonucleotide (i.e., not being part of the present invention), an antagonistic protein such as a fusion protein, the antibody and/or small molecule inhibits (antagonist) the identical or a different growth receptor or it inhibits (antagonist) a signal transduction factor. The growth receptor is for example TGF-beta receptor I (TβRI), TGF-beta receptor II (TβRII), or receptors for VEGF, HGF, PDGF and/or SEMA3 (Plexin). The signal transduction factor is for example p38MAPK, ERK1, ERK2, PI3K, Akt, NF-κB, pSMAD2, pSMAD3, Src, Pyk2, FAK and/or p-p130Cas.

An antibody in combination with the oligonucleotide or the pharmaceutical composition of the present invention is for example an anti-NRP1 antibody such as MNRP1685A (Genentech), a VEGF fusion protein such as Aflibercept and/or a bispecific antibody. A small molecule in combination with the oligonucleotide or the pharmaceutical composition of the present invention is for example EG00229 (Tocris). In case of an ophthalmic disease such as AMD or DME an oligonucleotide of the present invention may be combined with an anti-VEGF antibody or an antagonistic protein such as a fusion protein, laser therapy and/or a corticosteroid such as cortisol (C₂₁H₃₀O₅), corticosterone (C₂₁H₃₀O₄), cortisone (C₂₁H₂₈O₅) and/or aldosterone (C21H2805).

A subject of the present invention is for example a mammalian, a bird or a fish.

### EXAMPLES

The following examples illustrate different embodiments of the present invention, but the invention is not limited to these examples.

### Example 1: Design of Human, Mouse and rat NRP1 Antisense Oligonucleotides

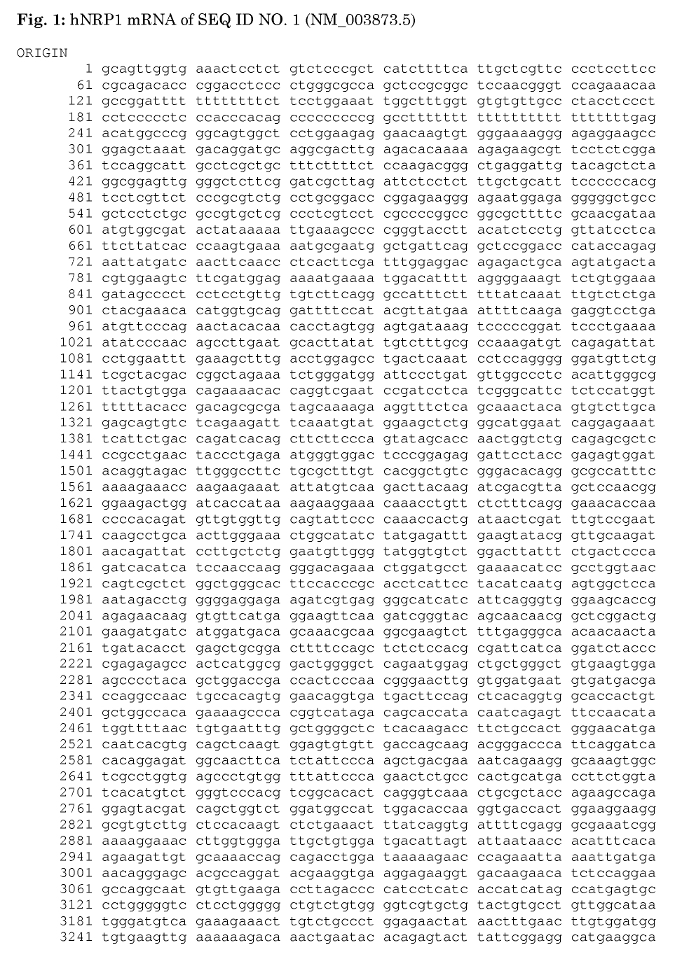

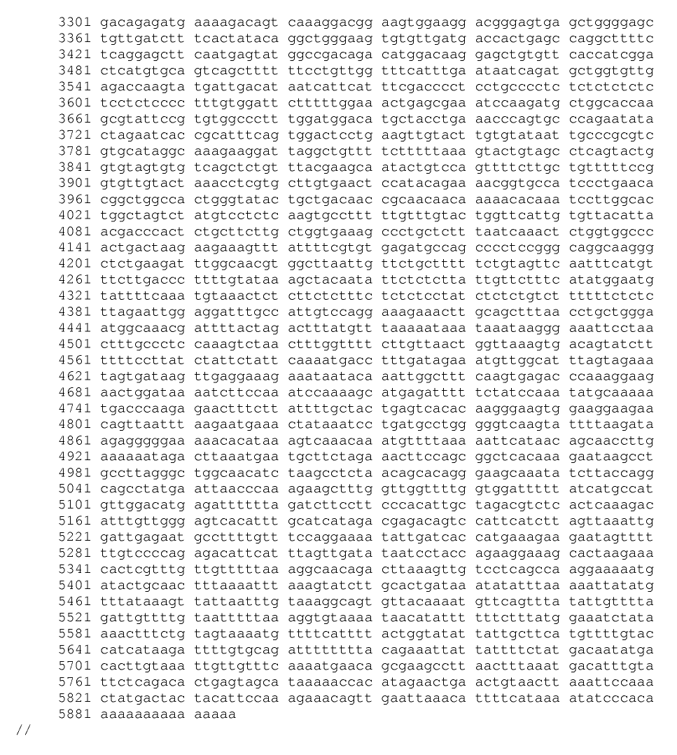

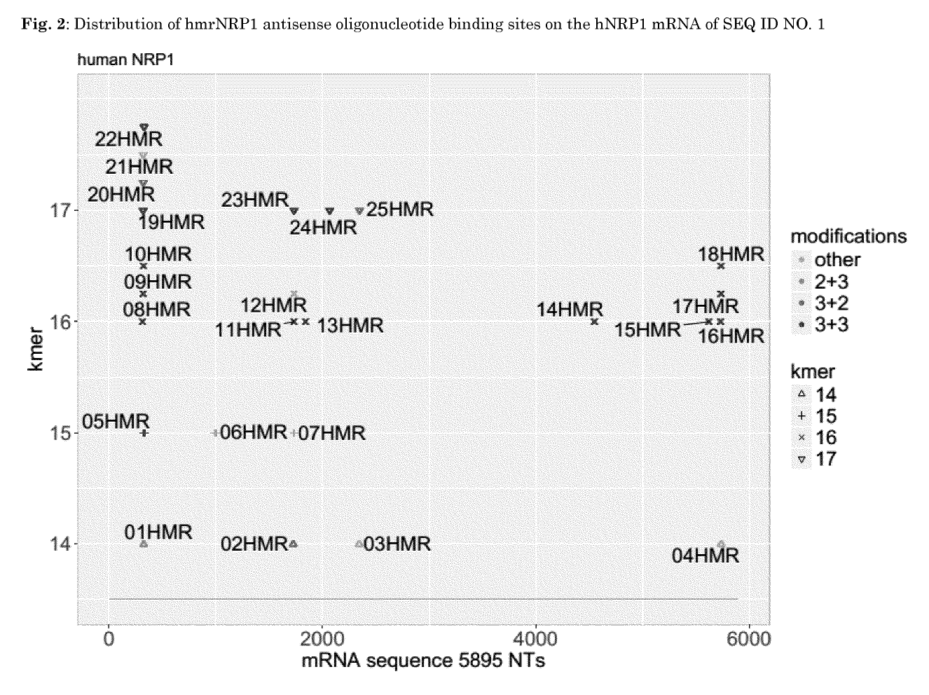

For the design of antisense oligonucleotides with specificity for human (h), mouse (m), rat (r) NRP1, the hNRP1 mRNA sequence with SEQ ID No. 1 (seq. ref. ID NM_003873.5; FIG. 1) was used. 14, 15, 16 and 17mers were designed according to in-house criteria, negl (described in WO2014154843 A1), scrambled 6 (S6) or scrambled 5 (S5) (Table 1) were used as control antisense oligonucleotides indicated in the respective experiments. The distribution of the antisense oligonucleotide binding sites on the hNRP1 mRNA is shown in FIG. 2.

### Example 2: Efficacy Screen of hmrNRP1 Antisense Oligonucleotides in Human and Mouse Cancer Cell Lines

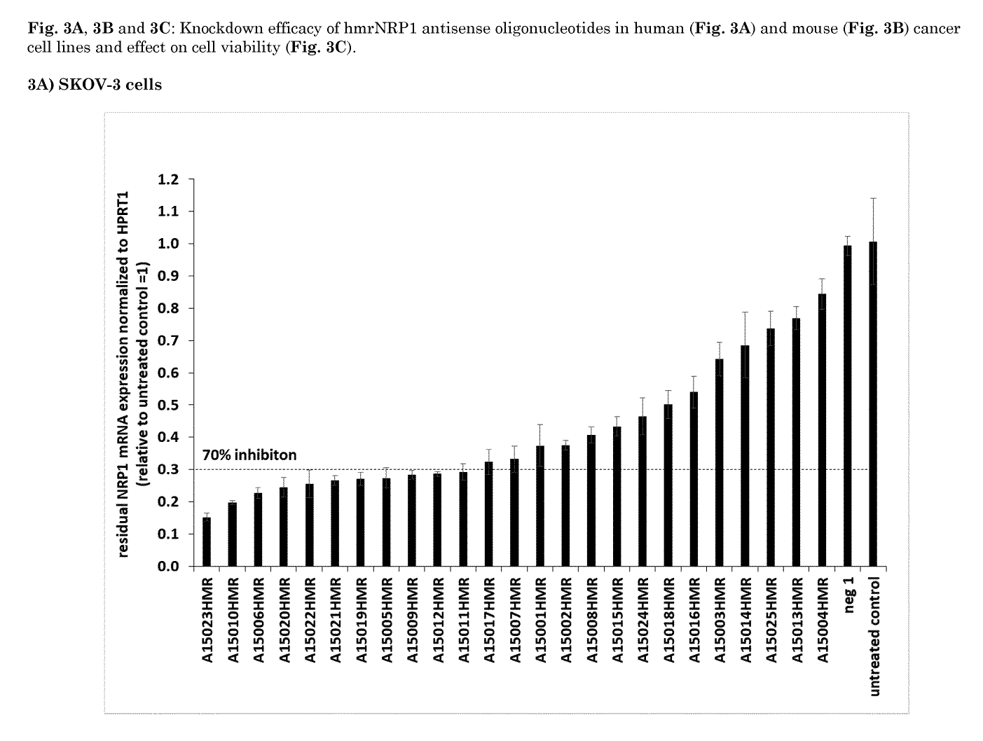

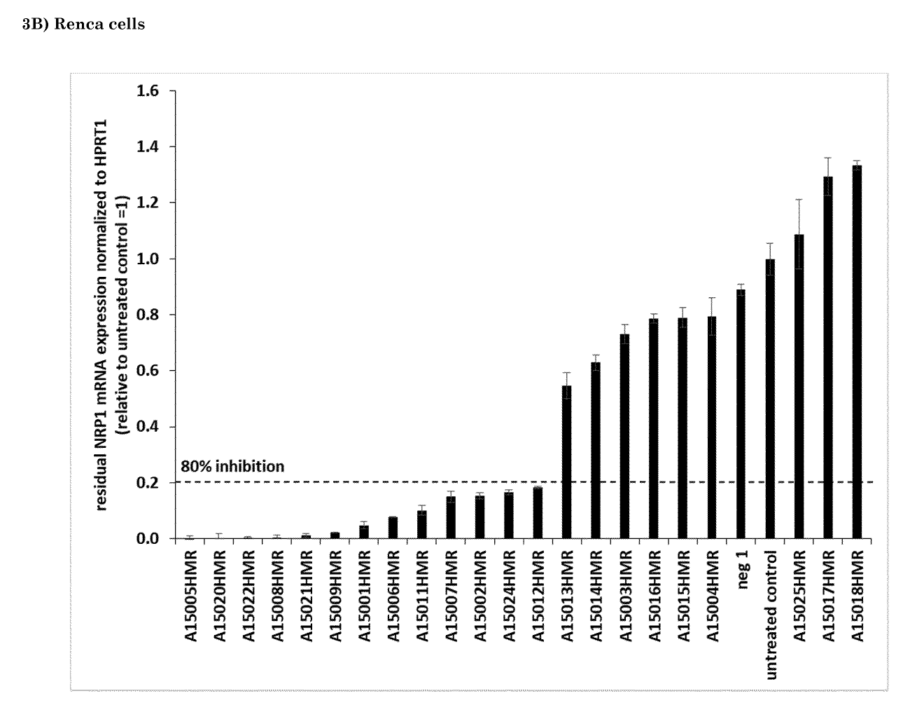

In order to analyze the efficacy of hmrNRP1 antisense oligonucleotides of the present invention with regard to the knockdown of human and mouse NRP1 mRNA expression in cancer cell lines, SKOV-3 (human ovary adenocarcinoma) and Renca (mouse renal cell carcinoma) cells were treated with a single concentration of 10 ∥M (without addition of any transfection reagent; this process is called gymnotic delivery) of the respective antisense oligonucleotide as shown in FIG. 3A and 3B. Human and mouse NRP1 and HPRT1 mRNA expression were analyzed after three days using the QuantiGene Singleplex assay (Affymetrix) and hmNRP1 expression values were normalized to HPRT1 values. Strikingly, as shown in FIG. 3A (SKOV-3 cells) and 3B (Renca cells), a knockdown efficiency of >80% was observed for 2 and 13 antisense oligonucleotides, respectively. Values of the mean normalized mRNA expression of hmNRP1 compared to non-treated cells are listed for SKOV-3 (Table 2) and Renca cells (Table 3) in the following:

TABLE 2
List of the mean normalized hNRP1 mRNA expression
values in antisense oligonucleotide-treated SKOV-3
cells compared to untreated cells.

| | Relative hNRP1 mRNA expression |
|---|---|
| Compound ID | (relative to untreated cells) |
| A15023HMR | 0.15 |
| A15010HMR | 0.20 |
| A15006HMR | 0.23 |
| A15020HMR | 0.24 |
| A15022HMR | 0.25 |
| A15021HMR | 0.26 |
| A15019HMR | 0.27 |
| A15005HMR | 0.27 |
| A15009HMR | 0.28 |
| A15012HMR | 0.28 |
| A15011HMR | 0.29 |
| A15017HMR | 0.32 |
| A15007HMR | 0.33 |
| A15001HMR | 0.37 |
| A15002HMR | 0.37 |
| A15008HMR | 0.40 |
| A15015HMR | 0.43 |
| A15024HMR | 0.46 |
| A15018HMR | 0.50 |
| A15016HMR | 0.54 |
| A15003HMR | 0.64 |
| A15014HMR | 0.68 |
| A15025HMR | 0.73 |
| A15013HMR | 0.76 |
| A15004HMR | 0.84 |
| neg 1 | 0.99 |

TABLE 3
List of the mean normalized mNRP1 mRNA expression
values in antisense oligonucleotide-treated Renca
cells compared to untreated cells.

| | Relative mNRP1 mRNA expression |
|---|---|
| Compound ID | (relative to untreated cells) |
| A15005HMR | 0.00 |
| A15020HMR | 0.00 |
| A15022HMR | 0.01 |
| A15008HMR | 0.01 |
| A15021HMR | 0.01 |
| A15009HMR | 0.02 |
| A15001HMR | 0.05 |
| A15006HMR | 0.08 |
| A15011HMR | 0.10 |
| A15007HMR | 0.15 |
| A15002HMR | 0.15 |
| A15024HMR | 0.17 |
| A15012HMR | 0.18 |
| A15013HMR | 0.55 |
| A15014HMR | 0.63 |
| A15003HMR | 0.73 |
| A15016HMR | 0.79 |
| A15015HMR | 0.79 |
| A15004HMR | 0.80 |
| neg1 | 0.89 |
| A15025HMR | 1.09 |
| A15017HMR | 1.30 |
| A15018HMR | 1.33 |

### Example 3: Correlation Analysis of Antisense Oligonucleotide Efficacy in Human SKOV-3 and Mouse Renca Cells

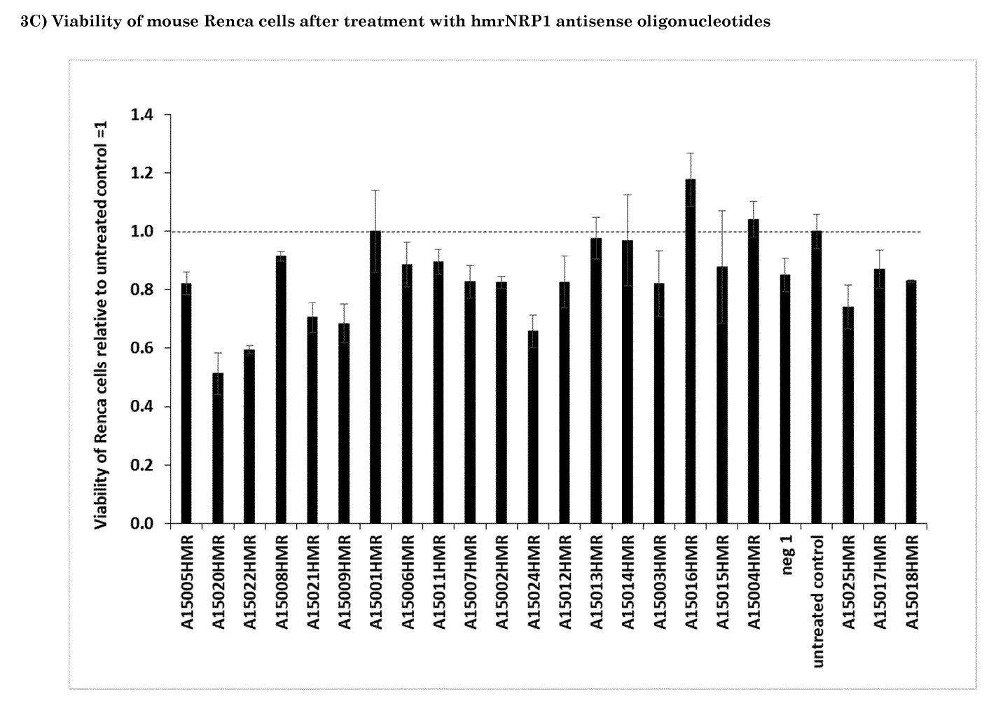

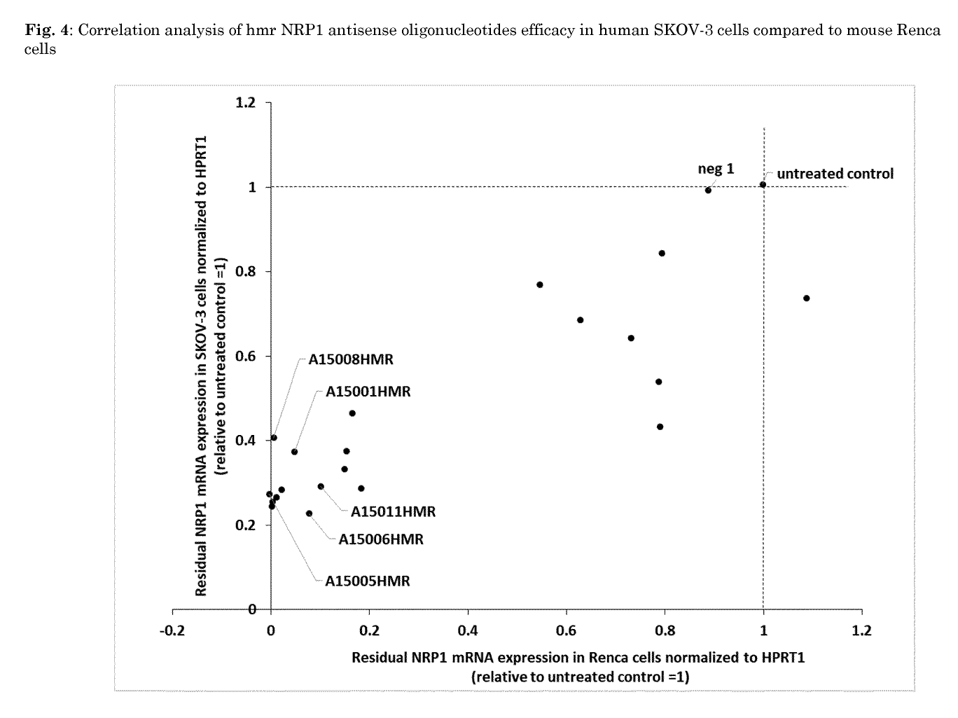

To further select the candidates with the highest activity in both tested cell lines, SKOV-3 and Renca cells, a correlation analysis was performed (data derived from FIG. 3A and 3B). As depicted in FIG. 4, 5 potent antisense oligonucleotides were selected for further investigation, namely A15001HMR (SEQ ID No. 3), A15005HMR (SEQ ID No. 2), A15006HMR (SEQ ID No. 4), A15008HMR (SEQ ID No. 5) and A15011HMR (SEQ ID No. 6). Importantly, the control antisense oligonucleotide negl had no negative influence on the expression of hmrNRP1 in both cell lines.

### Example 4: IC₅₀ Determination of Selected hmrNRP1 Antisense Oligonucleotides in Renca Cells (mRNA Level)

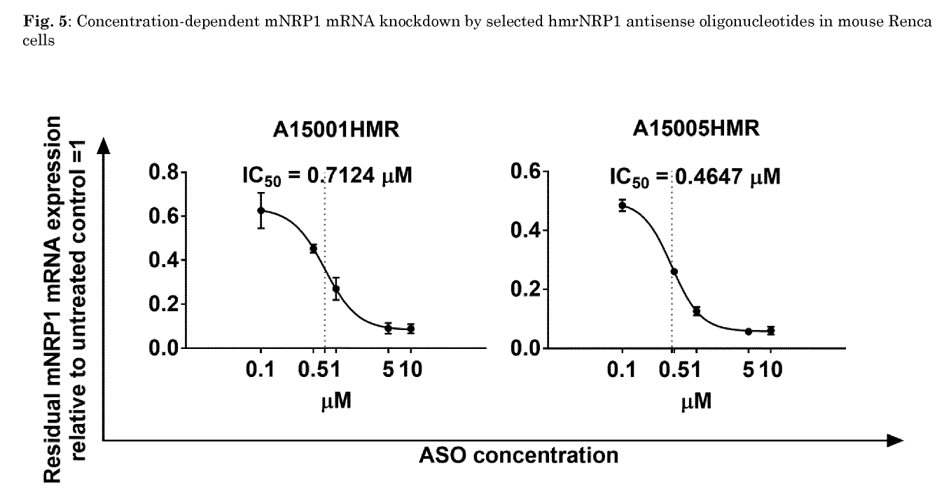

In order to determine the IC₅₀ of the hmrNRP1 antisense oligonucleotides A15005HMR (SEQ ID No. 2) and A15001HMR (SEQ ID No. 3), Renca cells were treated with the respective antisense oligonucleotide at concentrations of 10 ∥M, 5 μM, 1 μM, 500 nM and 100 nM, respectively. Mouse (m)NRP1 mRNA expression was analyzed three days after start of oligonucleotide treatment. As shown in FIG. 5 and following Table 4, the antisense oligonucleotides A15001HMR (SEQ ID No. 3) and A15005HMR (SEQ ID No. 2) had a high potency in Renca cells with regard to downregulation of mNRP1 mRNA compared to untreated cells with a maximal target inhibition of 90.2% and 92.8%, respectively. Table 4 shows IC₅₀ values and target inhibition of the above mentioned selected antisense oligonucleotides in Renca cells:

TABLE 4
Overview of IC₅₀ values for mNRP1 antisense oligonucleotides

| | Inhibition [%] NRP1 mRNA in Renca cells | | | | | |
|---|---|---|---|---|---|---|
| Compound ID | IC₅₀ [μM] | 10 μM | 5 μM | 1 μM | 0.5 μM | 0.1 μM |
| A15001HMR | 0.7124 | 90.19 | 89.08 | 66.13 | 48.21 | 27.37 |
| A15005HMR | 0.4647 | 92.80 | 93.01 | 84.37 | 70.31 | 40.34 |

### Example 5: Concentration-Dependent hNRP1 Protein Knockdown by A15001HMR (SEQ ID No. 3), A15005HMR (SEQ ID No. 2), A15006HMR (SEQ ID No. 4), A15008HMR (SEQ ID No. 5) and A15011HMR (SEQ ID No. 6)

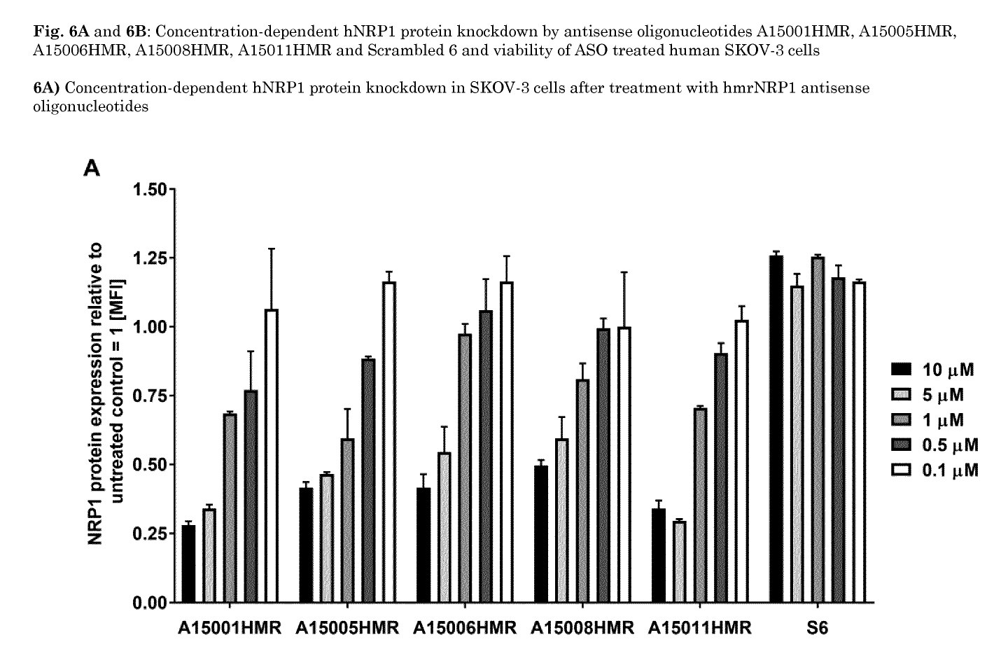

The highly potent hmrNRP1 antisense oligonucleotides A15001HMR (SEQ ID No. 3), A15005HMR (SEQ ID No. 2), A15006HMR (SEQ ID No. 4), A15008HMR (SEQ ID No. 5), A15011HMR (SEQ ID No. 6) were characterized in detail with regard to their knockdown efficacy on hNRP1 protein expression and their influence on cell viability at different concentrations in human SKOV-3 cells. SKOV-3 cells were therefore treated with different concentrations of the respective antisense oligonucleotide for 3 days, then medium was changed and fresh oligonucleotide was added at the respective concentrations for further 3 days. Protein expression was analyzed after a total treatment time of six days by flow cytometry using the anti-human NRP1 antibody (clone 12C2) and cell viability was analyzed using 7-AAD staining. As shown in FIG. 6A, A15001HMR (SEQ ID No. 3), A15005HMR (SEQ ID No. 2), and A15011HMR (SEQ ID No. 6) antisense oligonucleotides show potent and concentration-dependent inhibition of hNRP1 protein in SKOV-3 cells after 3+3 days, whereas treatment with S6 had no inhibitory effect. FIG. 6B depicts that oligonucleotide treatment had no major impact on cell viability. As shown in FIG. 6 and following Table 5, the antisense oligonucleotides A15001HMR (SEQ ID No. 3), A15005HMR (SEQ ID No. 2) and A15011HMR (SEQ ID No. 6) had the highest potency in SKOV-3 cells with regard to downregulation of hNRP1 protein compared to untreated cells with a maximal target inhibition of 71.99%, 58.44% and 65.78%, respectively. Table 5 summarizes protein knockdown efficiency of the selected human NRP1 antisense oligonucleotides A15001HMR (SEQ ID No. 3), A15005HMR (SEQ ID No. 2), A15006HMR (SEQ ID No. 4), A15008HMR (SEQ ID No. 5) and A15011HMR (SEQ ID No. 6) in SKOV-3 cells.

TABLE 5
Overview of protein knockdown efficiency of hmrNRP1 antisense
oligonucleotides in human SKOV-3 cells.

| | Inhibition [%] NRP1 protein in SKOV-3 cells | | | | |
|---|---|---|---|---|---|
| ASO | 10 μM | 5 μM | 1 μM | 0.5 μM | 0.1 μM |
| A15001HMR | 71.99 | 66.34 | 31.17 | 22.83 | 0.00 |
| A15005HMR | 58.44 | 53.60 | 40.14 | 11.61 | 0.00 |
| A15006HMR | 58.27 | 45.35 | 2.87 | 0.00 | 0.00 |
| A15008HMR | 50.52 | 40.67 | 18.88 | 0.50 | 0.00 |
| A15011HMR | 65.78 | 70.18 | 29.58 | 9.74 | 0.00 |

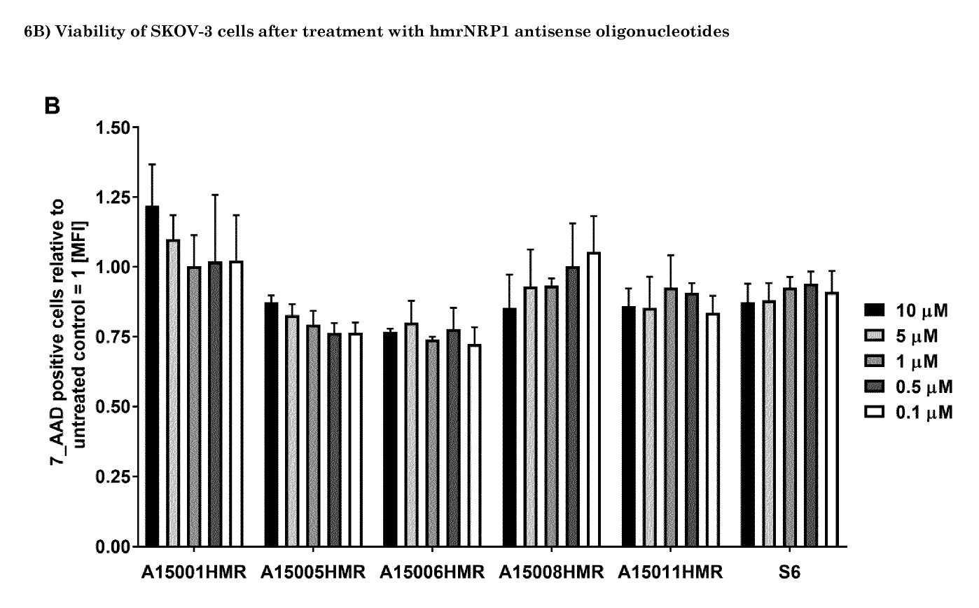

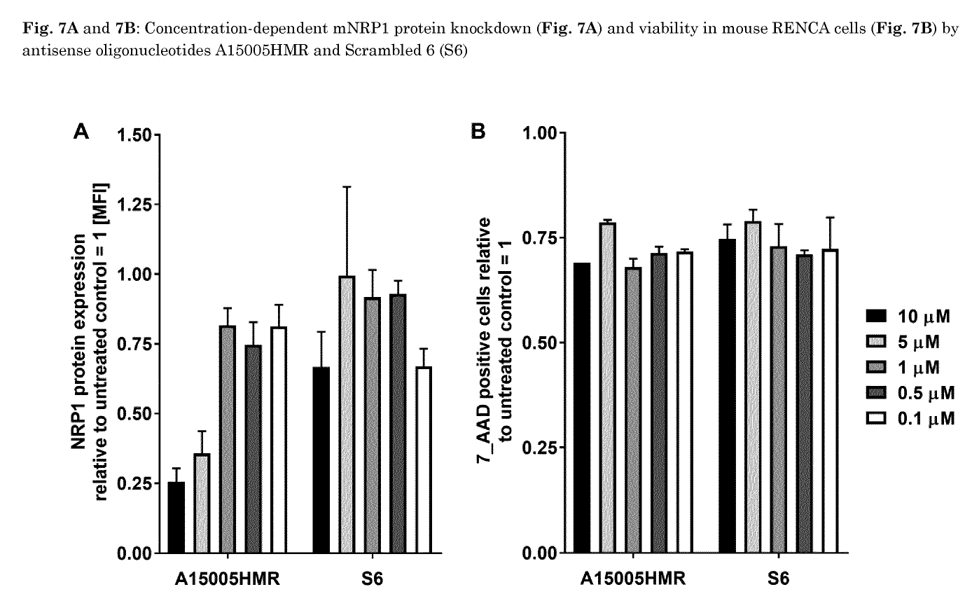

Furthermore, protein knockdown efficacy of A15005HMR (SEQ ID No. 2) was investigated in mouse cells. Therefore, mouse Renca cells were treated with different concentrations of A15005HMR (SEQ ID No. 2) and protein expression was analyzed after three days by flow cytometry using the anti-mouse NRP1 antibody (clone 3DS304M) and 7-AAD to investigate viability. A15005HMR (SEQ ID No. 2) shows potent concentration-dependent inhibition of mNRP1 protein expression in Renca cells after 3 days as depicted in FIG. 7A without affecting viability of Renca cells at any of the conditions tested, as shown in FIG. 7B.

### Example 6: Antisense Oligonucleotide-Mediated NRP1 mRNA Knockdown in Retinae of C57BL/6 Mice 3 and 10 Days After Treatment

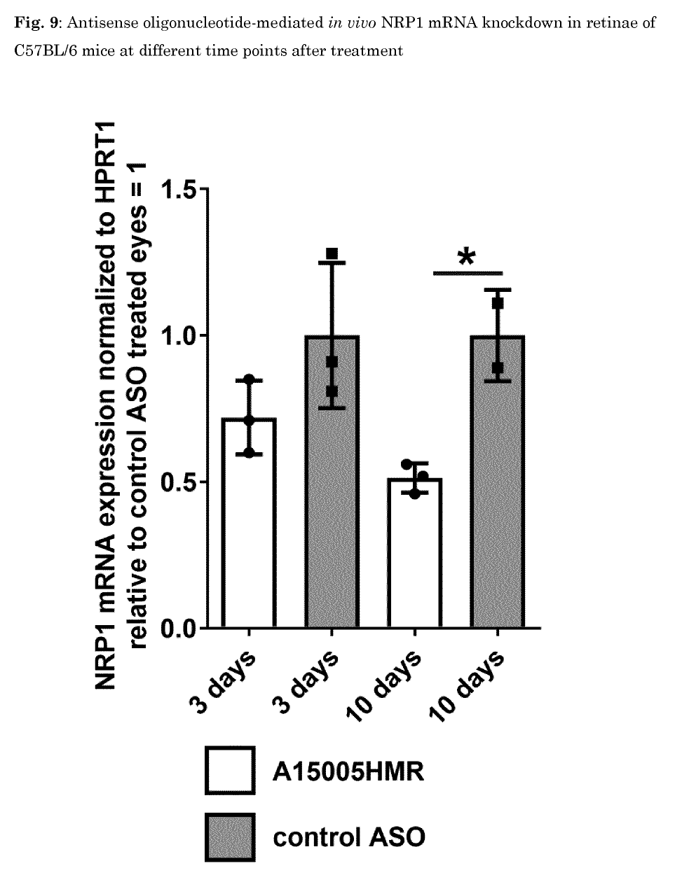

In vivo knockdown of NRP1 mRNA in retinae of C57BL/6 mice was analyzed 3 days and 10 days after single intravitreal injection of 1 μl of a 100 _{μ}M solution of the human-mouse cross-reactive antisense oligonucleotide A15005HMR (SEQ ID No. 2). As control, S5 (SEQ ID No. 29) an oligonucleotide having no sequence complementary to any human or mouse mRNA was injected into contralateral eyes. The results depicted in FIG. 9 revealed a significant knockdown (p=0.0172) of NRP1 mRNA in retinae of mice treated with A15005HMR compared to retinae of mice treated with control oligonucleotide S5. The expression values were normalized to expression values of the housekeeping gene HPRT1. This inhibitory effect was more pronounced after 10 days than after 3 days.

### Example 7: Antisense Oligonucleotide-Mediated NRP1 mRNA Knockdown in Retinae of C57BL/6 Mice 24 Days After Treatment

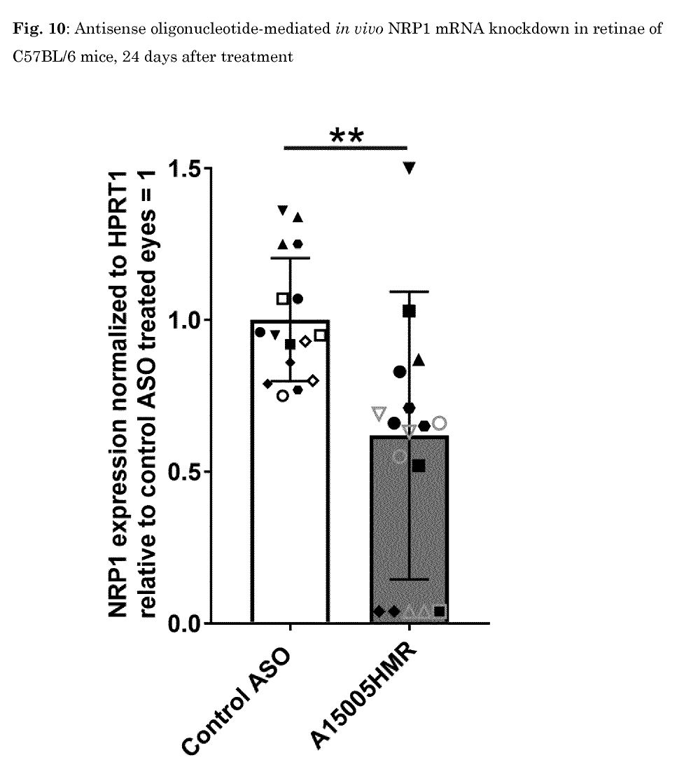

The results depicted in FIG. 9 revealed an efficient knock-down of NRP1 mRNA by A15005HMR in vivo, 10 days after single intravitreal injection. In order to investigate target knockdown at later time points, C57BL/6 mice were treated with A15005HMR (SEQ ID No. 2) or control antisense-oligonucleotide S5 (SEQ ID No. 29) by single intravitreal injections of 1 μl of a 100 ∥M oligonucleotide solution. Retinae were isolated 24 days later. The results depicted in FIG. 10 revealed a significant knockdown (p=0.0057) of retinal NRP1 mRNA levels by A15005HMR with a median reduction by ˜40% compared to control oligonucleotide S5. Expression values were normalized to expression values of the housekeeping gene HPRT1. Accordingly, these results indicate that A15005HMR significantly inhibits NRP1 mRNA expression in retinae for a duration of at least 24 days after single intravitreal injection in vivo.

1. An antisense oligonucleotide comprising 12 to 18 nucleotides, wherein at least one of the nucleotides is modified, and the oligonucleotide hybridizes with a nucleic acid sequence of human neuropilin 1 (NRP1, CD304) of SEQ ID NO.1 (NM_003873.5), wherein the oligonucleotide inhibits at least 50% of the NRP1 expression.

2. The oligonucleotide of claim 1, wherein the modified nucleotide is selected from the group consisting of a bridged nucleic acid such as LNA, cET, ENA, 2′Fluoro modified nucleotide, 2′O-Methyl modified nucleotide and a combination thereof.

3. The oligonucleotide of claim 1 or 2 hybridizing with NRP1 of SEQ ID NO.1 comprising a sequence selected from the group consisting of SEQ ID NO.2, SEQ ID NO.3, SEQ ID NO.4, SEQ ID NO.5, SEQ ID NO.6, SEQ ID NO.7, SEQ ID NO.8, SEQ ID NO.9, SEQ ID NO.10, SEQ ID NO.11, SEQ ID NO.12, SEQ ID NO.13, SEQ ID NO.14, SEQ ID NO.15, SEQ ID NO.16, SEQ ID NO.17, SEQ ID NO.18, SEQ ID NO.19, SEQ ID NO.20, SEQ ID NO.21, SEQ ID NO.22, SEQ ID NO.23, SEQ ID NO.24, SEQ ID NO.25, SEQ ID NO.26, and a combination thereof.

4. The oligonucleotide of any one of claims 1 to 3, wherein the oligonucleotide is selected from the group consisting of
(A15005HMR)
+G*+T*+C*T*C*A*A*G*T*C*G*C*+C*+T*+G,
(A15001HMR)
+A*+A*G*T*C*G*C*C*T*G*C*+A*+T*+C,
(A15006HMR)
+G*G*A*C*T*T*T*A*T*C*A*C*+T*C*+C,
(A15008HMR)
+A*+T*+C*C*T*G*T*C*A*T*T*T*A*+G*+C*+T,
(A15011HMR)
+T*+C*+G*G*A*C*A*A*A*T*C*G*A*+G*+T*+T,
(A15002HMR)
+A*+T*+C*G*A*G*T*T*A*T*C*A*+G*+T,
(A15003HMR)
+G*T*T*G*G*C*C*T*G*G*T*+C*G*+T,
(A15004HMR)
+G*+T*T*A*A*G*G*C*T*T*C*+G*C*+T,
(A15007HMR)
+T*C*+G*G*A*C*A*A*A*T*C*G*+A*+G*+T,
(A15009HMR)
+C*+G*+C*C*T*G*C*A*T*C*C*T*G*+T*+C*+A,
(A15010HMR)
+A*+G*+T*C*G*C*C*T*G*C*A*T*C*+C*+T*+G,
(A15012HMR)
+T*+T*+C*G*G*A*C*A*A*A*T*C*G*+A*G*+T,
(A15013HMR)
+A*+A*+T*A*A*G*T*C*C*A*G*A*C*+A*+C*+C,
(A15014HMR)
+T*+A*+C*T*G*T*C*A*C*T*T*T*A*+A*+C*+C,
(A15015HMR)
+G*+A*+A*G*C*A*A*T*A*A*T*A*T*+A*+C*+C,
(A15016HMR)
+G*+G*+C*T*T*C*G*C*T*G*T*T*C*+A*+T*+T,
(A15017HMR)
+T*+A*+A*G*G*C*T*T*C*G*C*T*G*+T*+T*+C,
(A15018HMR)
+T*+T*+A*A*G*G*C*T*T*C*G*C*T*+G*+T*+T,
(A15019HMR)
+C*+G*+C*C*T*G*C*A*T*C*C*T*G*T*+C*+A*+T,
(A15020HMR)
+T*+C*+G*C*C*T*G*C*A*T*C*C*T*G*T*+C*+A,
(A15021HMR)
+T*+C*G*C*C*T*G*C*A*T*C*C*T*G*T*C*+A,
(A15022HMR)
+G*+T*+C*T*C*A*A*G*T*C*G*C*C*T*+G*+C*+A,
(A15023HMR)
+T*+T*+C*G*G*A*C*A*A*A*T*C*G*A*+G*+T*+T,
(A15024HMR)
+C*+G*+A*T*C*T*T*G*A*A*C*T*T*C*+C*+T*+C,
(A15025HMR)
+T*+G*+G*C*A*G*T*T*G*G*C*C*T*G*G*+T*+C,
and a combination thereof, wherein + indicates an LNA nucleotide and * indicates a phosphorothioate (PTO) linkage between the nucleotides.

5. The oligonucleotide of any one of claims 1 to 4, wherein the oligonucleotide inhibits the expression of NRP1 at a nanomolar concentration.

6. A pharmaceutical composition comprising an antisense oligonucleotide of any one of claims 1 to 5 and a pharmaceutically acceptable carrier, excipient, diluent or a combination thereof.

7. The pharmaceutical composition of claim 6, further comprising a chemotherapeutic, another oligonucleotide, an antagonistic protein, an antibody and/or a small molecule being effective in treating a tumor or an ophthalmic disease.

8. The pharmaceutical composition of claim 6 or 7 or the antisense oligonucleotide according to any one of claims 1 to 5 further inhibiting the activity of a receptor such as a growth receptor selected from the group consisting of TGF-beta receptor I (TβRI), TGF-beta receptor II (TβRII), VEGF, HGF, PDGF and SEMA3 (Plexin), or a combination thereof.

9. The pharmaceutical composition of claim 6 or 7 or the antisense oligonucleotide according to any one of claims 1 to 5 further inhibiting the activity of a signal transduction factor such as p38MAPK, ERK1, ERK2, PI3K, Akt, NF-κB, pSMAD2, pSMAD3, Src, Pyk2, FAK, p-p130Cas, or a combination thereof.

10. The pharmaceutical composition of any one of claims 6 to 9 or the antisense oligonucleotide according to any one of claims 1 to 5 inhibiting the immigration of a Tre_{g} cell into a tumor.

11. The pharmaceutical composition of any one of claims 6 to 9, wherein the other oligonucleotide, the antagonistic protein, the antibody and/or the small molecule inhibits the identical or a different growth receptor or signal transduction factor than the antisense oligonucleotide according to any one of claims 1 to 5.

12. The pharmaceutical composition of any one of claims 6 to 11 or the antisense oligonucleotide according to any one of claims 1 to 5 for use in preventing and/or treating cancer, an ophthalmic disease, an autoimmune disorder and/or an immune disorder.

13. The pharmaceutical composition or the antisense oligonucleotide for use according to claim 12, wherein the ophthalmic disease is an angiogenic eye disease such as age related macular disease (AMD), diabetic retinopathy (DME), retinopathy of prematurity (Retinopathia praematurorum) or corneal neovascularization.

14. The pharmaceutical composition or the antisense oligonucleotide for use according to claim 12, wherein the cancer is bladder carcinoma, breast cancer, colorectal carcinoma, lung cancer, malignant melanoma, mesothelioma, lymphoma, skin cancer, bone cancer, prostate cancer, hepatocarcinoma, brain cancer, cancer of the larynx, liver, gall bladder, pancreas, testicular, rectum, parathyroid, thyroid, adrenal, neural tissue, head and neck, colon, stomach, bronchi, kidneys, basal cell carcinoma, neuroblastoma, squamous cell carcinoma, metastatic skin carcinoma, osteo sarcoma, Ewing's sarcoma, reticulum cell sarcoma, liposarcoma, leukemia, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm's tumor, seminoma, ovarian tumor, leiomyomater tumor, cervical dysplasia, retinoblastoma, soft tissue sarcoma, malignant carcinoid, topical skin lesion, rhabdomyosarcoma, Kaposi's sarcoma, osteogenic sarcoma, malignant hypercalcemia, renal cell tumor, polycythaemia vera, adenocarcinoma, anaplastic astrocytoma, glioblastoma multiforma, leukemia, or epidermoid carcinoma.

15. The pharmaceutical composition or the antisense oligonucleotide for use according to any one of claims 12 to 14, wherein the oligonucleotide or the composition is administrable locally or systemically.


SEQUENCE LISTING (29 sequences)

SEQ ID NO: 1 - DNA, 5895 bp, Homo sapiens
gcagttggtgaaactcctctgtctcccgctcatcttttcattgctcgttcccctccttcccgcagacacccggacctcccctgggcgccagctccgcggctccaacgggtccagaaacaagccggattttttttttttcttcctggaaattggctttggtgtgtgttgccctacctccctcctccccctcccacccacagcccccccccggccttttttttttttttttttttttttgagacatggcccgggcagtggctcctggaagaggaacaagtgtgggaaaagggagaggaagccggagctaaatgacaggatgcaggcgacttgagacacaaaaagagaagcgttcctctcggatccaggcattgcctcgctgctttcttttctccaagacgggctgaggattgtacagctctaggcggagttggggctcttcggatcgcttagattctcctctttgctgcatttccccccacgtcctcgttctcccgcgtctgcctgcggacccggagaagggagaatggagagggggctgccgctcctctgcgccgtgctcgccctcgtcctcgccccggccggcgcttttcgcaacgataaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggggacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaccccatcctcatcaccatcatagccatgagtgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatgaaggcagacagagatgaaaagacagtcaaaggacggaagtggaaggacgggagtgagctggggagctgttgatctttcactatacaggctgggaagtgtgttgatgaccactgagccaggcttttctcaggagcttcaatgagtatggccgacagacatggacaaggagctgtgttcaccatcggactcatgtgcagtcagcttttttcctgttggtttcatttgaataatcagatgctggtgttgagaccaagtatgattgacataatcattcatttcgacccctcctgcccctctctctctctctcctctcccctttgtggattctttttggaaactgagcgaaatccaagatgctggcaccaagcgtattccgtgtggccctttggatggacatgctacctgaaacccagtgcccagaatatactagaatcaccgcatttcagtggactcctgaagttgtacttgtgtataattgcccgcgtcgtgcataggcaaagaaggattaggctgttttctttttaaagtactgtagcctcagtactggtgtagtgtgtcagctctgtttacgaagcaatactgtccagttttcttgctgtttttccggtgttgtactaaacctcgtgcttgtgaactccatacagaaaacggtgccatccctgaacacggctggccactgggtatactgctgacaaccgcaacaacaaaaacacaaatccttggcactggctagtctatgtcctctcaagtgcctttttgtttgtactggttcattgtgttacattaacgacccactctgcttcttgctggtgaaagccctgctctttaatcaaactctggtggcccactgactaagaagaaagtttattttcgtgtgagatgccagcccctccgggcaggcaagggctctgaagatttggcaacgtggcttaattgttctgctttttctgtagttcaatttcatgtttcttgacccttttgtataaagctacaatattctctcttattgttctttcatatggaatgtattttcaaatgtaaactctcttctctttctctctcctatctctctgtcttttttctctcttagaattggaggatttgccattgtccaggaaagaaacttgcagctttaacctgctgggaatggcaaacgattttactagactttatgtttaaaaataaataaataagggaaattcctaactttgccctccaaagtctaactttggttttcttgttaactggttaaagtgacagtatcttttttccttatctattctattcaaaatgacctttgatagaaatgttggcatttagtagaaatagtgataagttgaggaaagaaataatacaaattggctttcaagtgagacccaaaggaagaactggataaaatcttccaaatccaaaagcatgagatttttctatccaaatatgcaaaaatgacccaagagaactttcttattttgctactgagtcacacaagggaagtggaaggaagaacagttaatttaagaatgaaactataaatcctgatgcctgggggtcaagtattttaagataagagggggaaaaacacataaagtcaaacaaatgttttaaaaattcataacagcaaccttgaaaaaatagacttaaatgaatgcttctagaaacttccagcggctcacaaagaataagcctgccttagggctggcaacatctaagcctctaacagcacagggaagcaaatatcttaccaggcagcctatgaattaacccaaagaagctttggttggttttggtggatttttatcatgccatgttggacatgagattttttagatcttccttcccacattgctagacgtctcactcaaagacatttgttgggagtcacatttgcatcatagacgagacagtccattcatcttagttaaattggattgagaatgccttttgtttccaggaaaatattgatcaccatgaaagaagaatagttttttgtccccagagacattcatttagttgatataatcctaccagaaggaaagcactaagaaacactcgtttgttgtttttaaaggcaacagacttaaagttgtcctcagccaaggaaaaatgatactgcaactttaaaatttaaagtatcttgcactgataaatatatttaaaaattatatgtttataaagttattaatttgtaaaggcagtgttacaaaatgttcagtttatattgttttagattgttttgtaatttttaaaggtgtaaaataacatattttttctttatggaaatctataaaactttctgtagtaaaatgttttcattttactggtatattattgcttcatgttttgtaccatcataagattttgtgcagattttttttacagaaattattattttctatgacaatatgacacttgtaaattgttgtttcaaaatgaacagcgaagccttaactttaaatgacatttgtattctcagacactgagtagcataaaaaccacatagaactgaactgtaacttaaattccaaactatgactactacattccaaagaaacagttgaattaaacattttcataaaatatcccacaaaaaaaaaaaaaaaa

SEQ ID NO: 2 - DNA, 15 bp, Artificial Sequence
  antisense oligonucleotide
gtctcaagtcgcctg

SEQ ID NO: 3 - DNA, 14 bp, Artificial Sequence
  antisense oligonucleotide
aagtcgcctgcatc

SEQ ID NO: 4 - DNA, 15 bp, Artificial Sequence
  antisense oligonucleotide
ggactttatcactcc

SEQ ID NO: 5 - DNA, 16 bp, Artificial Sequence
  antisense oligonucleotide
atcctgtcatttagct

SEQ ID NO: 6 - DNA, 16 bp, Artificial Sequence
  antisense oligonucleotide
tcggacaaatcgagtt

SEQ ID NO: 7 - DNA, 14 bp, Artificial Sequence
  antisense oligonucleotide
atcgagttatcagt

SEQ ID NO: 8 - DNA, 14 bp, Artificial Sequence
  antisense oligonucleotide
gttggcctggtcgt

SEQ ID NO: 9 - DNA, 14 bp, Artificial Sequence
  antisense oligonucleotide
gttaaggcttcgct

SEQ ID NO: 10 - DNA, 15 bp, Artificial Sequence
  antisense oligonucleotide
tcggacaaatcgagt

SEQ ID NO: 11 - DNA, 16 bp, Artificial Sequence
  antisense oligonucleotide
cgcctgcatcctgtca

SEQ ID NO: 12 - DNA, 16 bp, Artificial Sequence
  antisense oligonucleotide
agtcgcctgcatcctg

SEQ ID NO: 13 - DNA, 16 bp, Artificial Sequence
  antisense oligonucleotide
ttcggacaaatcgagt

SEQ ID NO: 14 - DNA, 16 bp, Artificial Sequence
  antisense oligonucleotide
aataagtccagacacc

SEQ ID NO: 15 - DNA, 16 bp, Artificial Sequence
  antisense oligonucleotide
tactgtcactttaacc

SEQ ID NO: 16 - DNA, 16 bp, Artificial Sequence
  antisense oligonucleotide
gaagcaataatatacc

SEQ ID NO: 17 - DNA, 16 bp, Artificial Sequence
  antisense oligonucleotide
ggcttcgctgttcatt

SEQ ID NO: 18 - DNA, 16 bp, Artificial Sequence
  antisense oligonucleotide
taaggcttcgctgttc

SEQ ID NO: 19 - DNA, 16 bp, Artificial Sequence
  antisense oligonucleotide
ttaaggcttcgctgtt

SEQ ID NO: 20 - DNA, 17 bp, Artificial Sequence
  antisense oligonucleotide
cgcctgcatcctgtcat

SEQ ID NO: 21 - DNA, 17 bp, Artificial Sequence
  antisense oligonucleotide
tcgcctgcatcctgtca

SEQ ID NO: 22 - DNA, 17 bp, Artificial Sequence
  antisense oligonucleotide
tcgcctgcatcctgtca

SEQ ID NO: 23 - DNA, 17 bp, Artificial Sequence
  antisense oligonucleotide
gtctcaagtcgcctgca

SEQ ID NO: 24 - DNA, 17 bp, Artificial Sequence
  antisense oligonucleotide
ttcggacaaatcgagtt

SEQ ID NO: 25 - DNA, 17 bp, Artificial Sequence
  antisense oligonucleotide
cgatcttgaacttcctc

SEQ ID NO: 26 - DNA, 17 bp, Artificial Sequence
  antisense oligonucleotide
tggcagttggcctggtc

SEQ ID NO: 27 - DNA, 18 bp, Artificial Sequence
  antisense oligonucleotide negative control
cgtttaggctatgtactt

SEQ ID NO: 28 - DNA, 17 bp, Artificial Sequence
  antisense oligonucleotide negative control scrambled 6
tctatcgtgatgtttct

SEQ ID NO: 29 - DNA, 17 bp, Artificial Sequence
  antisense oligonucleotide negative control scrambled 5
ttatgtccggttatttc